(19) United States
(12) Patent Application Publication
(10) Pub. No.: US 2011/0288316 A1
(43) Pub. Date: Nov. 24, 2011

(54) METHOD FOR PRODUCING PROPYLENE OXIDE

(75) Inventors: Tomonori Kawabata, Toyonaka-shi (JP); Tetsuro Yonemoto, Toyonaka-shi (JP)

(73) Assignee: SUMITOMO CHEMICAL COMPANY, LIMITED, Chuo-ku, Tokyo (JP)

(21) Appl. No.: 13/147,110

(22) PCT Filed: Jan. 29, 2010
(86) PCT No.: PCT/JP2010/051656

§ 371 Date: Jul. 29, 2011

(30) Foreign Application Priority Data
Feb. 2, 2009 (JP) 2009-021274

(51) Int. Cl. C07D 301/06

(52) U.S. Cl. 549/533

(57) ABSTRACT

A method for producing propylene oxide which comprises reacting propylene, oxygen and hydrogen, in the presence of a titanosilicate and a noble metal supported on silylated active carbon, in a solvent.

### TECHNICAL FIELD

The present invention relates to a method for producing propylene oxide.

### BACKGROUND ART

A method for producing propylene oxide from propylene, oxygen, and hydrogen is known which uses titanosilicate and a noble metal supported by active carbon having a particular total pore volume as catalysts (see e.g., Japanese Patent Laid-Open No. 2008-201776).

### SUMMARY OF INVENTION

The present invention provides a method for producing propylene oxide efficiently.

The present application relates to the following inventions.

[1] A method for producing propylene oxide which comprises reacting propylene, oxygen and hydrogen, in the presence of a titanosilicate and a noble metal supported on silylated active carbon, in a solvent.
[2] The production method according to [1], wherein the titanosilicate has a pore composed of 12- or more membered oxygen ring.
[3] The production method according to [1], wherein the titanosilicate is a crystalline titanosilicate having an MWW structure, or a Ti-MWW precursor.
[4] The production method according to [1], wherein the titanosilicate has an X-ray diffraction pattern reproduced in the form of interplanar spacings d of
1.24±0.08 nm,
1.08±0.03 nm,
0.9±0.03 nm,
0.6±0.03 nm,
0.39±0.01 nm, and
0.34±0.01 nm.
   [5] The production method according to any one of [1] to [4], wherein the solvent comprises an organic solvent.
   [6] The production method according to [5], wherein the organic solvent comprises at least one selected from the group consisting of alcohol, ketone, nitrile, ether, aliphatic hydrocarbon, aromatic hydrocarbon, halogenated hydrocarbon, and ester.
   [7] The production method according to [5] or [6], wherein the organic solvent is acetonitrile.
   [8] The production method according to any one of [5] to [7], wherein the solvent is a mixture of an organic solvent and water having an organic solvent-to-water weight ratio of 90:10 to 0.01:99.99.
   [9] The production method according to any one of [1] to [8], wherein the solvent contains a salt having an ammonium, alkylammonium, or alkylarylammonium ion.
   [10] The production method according to [9], wherein the salt comprises 1) an anion selected from sulfate ion, hydrogen sulfate ion, carbonate ion, hydrogen carbonate ion, phosphate ion, hydrogen phosphate ion, dihydrogen phosphate ion, hydrogen pyrophosphate ion, pyrophosphate ion, halogen ion, nitrate ion, hydroxide ion, and carboxylate ions having 1 to 10 carbon atoms and 2) a cation selected from ammonium ion, alkylammonium ions, and alkylarylammonium ions.
   [11] The production method according to any one of [1] to [10], wherein the solvent contains a quinoid compound or a dihydro form thereof.
   [12] The production method according to [11], wherein the quinoid compound is a phenanthraquinone compound or a compound represented by formula (1):

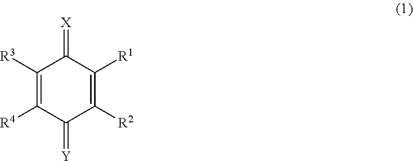

wherein R¹ and R² each independently represent a hydrogen atom, or R¹ and R² are combined, together with their carbon atoms to which R¹ and R² are bonded, to represent a benzene ring that may be substituted with an alkyl or hydroxyl group or a naphthalene ring that may be substituted with an alkyl or hydroxyl group; R³ and R⁴ are combined, together with their carbon atoms to which R³ and R⁴ are bonded, to represent a benzene ring that may be substituted with an alkyl or hydroxyl group or a naphthalene ring that may be substituted with an alkyl or hydroxyl group; and X and Y each independently represent an oxygen atom or an NH group.
[13] The production method according to [12], wherein the quinoid compound is a phenanthraquinone compound or a compound represented by formula (2):

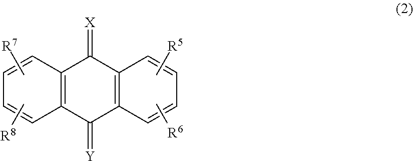

wherein X and Y each independently represent an oxygen atom or an NH group; and R⁵, R⁶, R⁷, and R⁸ each independently represent a hydrogen atom, a hydroxyl group, or an alkyl group.
[14] The production method according to [12], wherein X and Y are oxygen atoms.

### BRIEF DESCRIPTION OF DRAWINGS

FIG. 1 is a graph showing a ¹H-²⁹Si MAS NMR spectrum of washed active carbon;

FIG. 2 is a graph showing a ¹H-²⁹Si MAS NMR spectrum of silylated active carbon (I);

FIG. 3 is a graph showing a ¹H-²⁹Si MAS NMR spectrum of silylated active carbon (II);

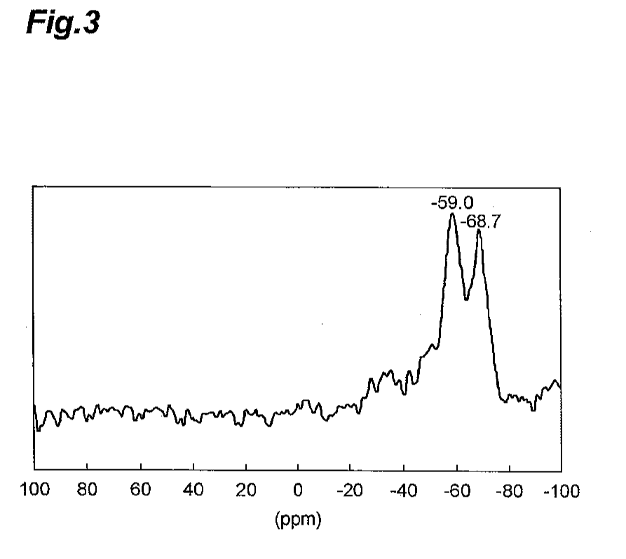

FIG. 4 is a graph showing a ²⁹Si MAS NMR spectrum of dimethyldichlorosilane;

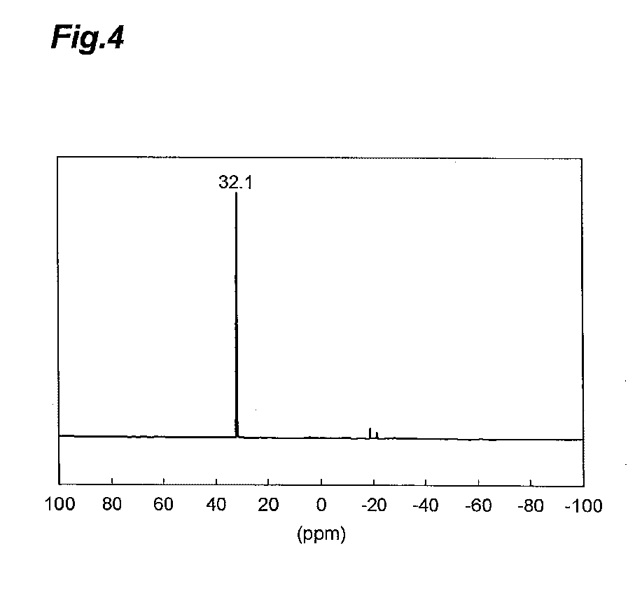

FIG. 5 is a graph showing a ²⁹Si MAS NMR spectrum of octyltrichlorosilane;

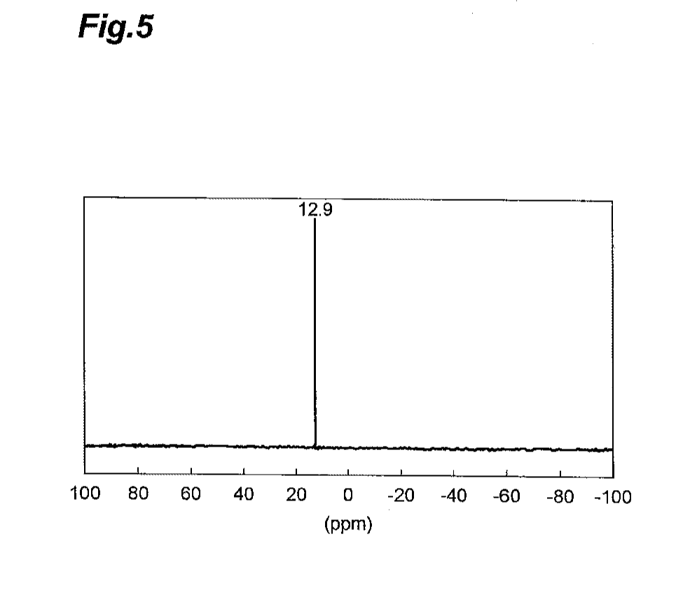

### DESCRIPTION OF EMBODIMENTS

Hereinafter, the present invention will be described in detail.

In the present invention, reaction of propylene, oxygen, and hydrogen is performed in the presence of a titanosilicate and a noble metal supported on silylated active carbon.

The production method of the present invention is excellent in the efficiency of the reaction of propylene, oxygen, and hydrogen, because the reaction is performed in the presence of a titanosilicate and a noble metal supported on silylated active carbon.

The silylated active carbon according to the present invention can be obtained by contacting an active carbon with a silylating agent.

The active carbon may have any shape such as powdery, granular, crushed, fibrous, and honeycomb shapes without limitations.

The active carbon to be contacted with a silylating agent is obtained by activating a raw material such as wood, sawdust, coconut shell, coal, or petroleum by a method conventionally known in the art.

The activation can be performed, for example, by a method comprising treating the raw material at a high temperature in the presence of water vapor, carbon dioxide, air, or the like or a method comprising treating the raw material with a chemical such as zinc chloride. The active carbon is preferably activated with the chemical.

Any silane-containing compound that exhibits the action of converting active hydrogen to a silyl group can be used as the silylating agent without particular limitations.

The silylating agent is represented by, for example, the following formula (I):

(R)ₙ—Si—(X)₄₋ₙ (I)

wherein n represents an integer of 0 to 3; R is a hydrocarbyl group having 1 to 20 carbon atoms; and X is halogen, a hydrogen atom, a hydrocarbyloxy group, or a silylimino group, wherein when n is 2 or 3, a plurality of the R moieties may be the same or different from each other.

Examples of the hydrocarbyloxy group include alkoxy, cycloalkyloxy, cycloalkylalkoxy, aryloxy, and aralkyloxy groups.

Examples of the alkoxy group include alkoxy groups having 1 to 20 carbon atoms, such as methoxy and ethoxy groups.

Examples of the cycloalkyloxy group include cycloalkyloxy groups having 3 to 10 carbon atoms, such as cyclopropyloxy and cyclohexyloxy groups.

Examples of the cycloalkylalkoxy group include cycloalkylalkoxy groups having 4 to 10 carbon atoms, such as cyclopropylmethoxy and cyclohexylmethoxy groups.

Examples of the aryloxy group include phenoxy groups.

Examples of the aralkyloxy group include benzyloxy groups.

The silylimino group may be substituted with an alkyl or cycloalkyl group, and examples thereof include trimethylsilylimino groups.

Examples of the halogen include chlorine, bromine, and iodine atoms.

The X moiety is preferably halogen.

Examples of the hydrocarbyl group include alkyl, cycloalkyl, cycloalkylalkyl, alkenyl, aryl, and aralkyl groups.

Examples of the alkyl group include C1 to C20 alkyl groups such as methyl, ethyl, n-propyl, n-butyl, n-hexyl, n-decyl, n-dodecyl, and n-octadecyl.

Examples of the cycloalkyl group include C3 to C10 cycloalkyl groups such as cyclohexyl groups.

Examples of the alkenyl group include C2 to C8 alkenyl groups such as vinyl and allyl groups.

Examples of the aryl group include C6 to C10 aryl groups such as phenyl and naphthyl groups.

Examples of the aralkyl group include C6 to C10 aralkyl groups such as phenylmethyl and phenylethyl groups.

The R moiety is preferably alkyl, cycloalkyl, aryl, and vinyl groups, more preferably an alkyl group.

The silylating agent is more preferably a silylating agent wherein R is at least one group selected from the group consisting of alkyl, cycloalkyl, aryl, and vinyl groups, and X is halogen.

Specific examples of the silylating agent include: halogen-containing silane compounds such as methyltrichlorosilane, dimethyldichlorosilane, trimethylchlorosilane, ethyltrichlorosilane, diethyldichlorosilane, triethylchlorosilane, n-propyltrichlorosilane, n-butyltrichlorosilane, n-hexyltrichlorosilane, n-decyltrichlorosilane, n-dodecyltrichlorosilane, n-octadecyltrichlorosilane, cyclohexylmethyldichlorosilane, phenyltrichlorosilane, methylphenyldichlorosilane, and diphenyldichlorosilane; and alkoxysilane compounds such as methyltrimethoxysilane, dimethyldimethoxysilane, trimethylmethoxysilane, ethyltrimethoxysilane, diethyldimethoxysilane, triethylmethoxysilane, methyltriethoxysilane, dimethyldiethoxysilane, trimethylethoxysilane, ethyltriethoxysilane, diethyldiethoxysilane, triethylethoxysilane, n-propyltrimethoxysilane, n-butyltrimethoxysilane, n-hexyltrimethoxysilane, n-hexyltriethoxysilane, n-octyltrimethoxysilane, n-octyltriethoxysilane, n-decyltrimethoxysilane, n-dodecyltriethoxysilane, n-octadecyltrimethoxysilane, cyclohexylmethyldimethoxysilane, phenyltrimethoxysilane, methylphenyldimethoxysilane, diphenyldimethoxysilane, phenyltriethoxysilane, diphenyldiethoxysilane, vinyltrimethoxysilane, and vinyltriethoxysilane.

These silylating agents may be used alone or in combination of two or more thereof. Preferable examples of the silylating agent include: silane compounds having a C1 to C20 alkyl group and chlorine, such as methyltrichlorosilane, dimethyldichlorosilane, trimethylchlorosilane, ethyltrichlorosilane, diethyldichlorosilane, triethylchlorosilane, n-propyltrichlorosilane, n-butyltrichlorosilane, n-hexyltrichlorosilane, n-octyltrichlorosilane, n-decyltrichlorosilane, n-dodecyltrichlorosilane, n-octadecyltrichlorosilane, and cyclohexylmethyldichlorosilane; and silane compounds having a C6 to C10 phenyl group and chlorine, such as phenyltrichlorosilane, methylphenyldichlorosilane, diphenyldichlorosilane, phenyltrichlorosilane, and diphenyldichlorosilane.

The active carbon may support the noble metal.

The active carbon is preferably subjected in advance to, for example, heat treatment at approximately 150° C. to 300° C. to remove adsorbed water prior to the contact of the active carbon with the silylating agent. This removal of adsorbed water from the active carbon can suppress the decomposition of the silylating agent which is generally unstable to moisture.

For the contact of the active carbon with the silylating agent, the silylating agent may be in any form such as vapor, liquid, and solution forms.

The contact is preferably performed at a temperature of 80 to 450° C. for the silylating agent in a vapor form. In the contact, a pressure is preferably approximately 0 to 10 MPa in terms of gage pressure for the silylating agent in a vapor form.

The contact of the active carbon with the vapor of the silylating agent can be performed, for example, by introducing the vapor of the silylating agent into a tightly closed container containing the active carbon and then keeping it for a predetermined time.

The introduction of the vapor of the silylating agent is usually performed under reduced pressure in the tightly closed container. The vapor of the silylating agent can be kept for a time appropriately set according to the amount of the active carbon or the kind of the silylating agent.

The silylating agent is generally contacted, with the active carbon, in an amount of, for example, but not particularly limited to, 0.001 to 50 parts by weight, preferably 0.1 to 5 parts by weight, per weight of the active carbon.

When the silylating agent is in a liquid form, this silylating agent may be used directly in the contact.

When the silylating agent in a solution form is contacted with the active carbon, the solution of the silylating agent can be prepared, for example, by dissolving the silylating agent in an organic solvent such as ethanol or toluene.

The contact of the silylating agent in a liquid or solution form with the active carbon can be performed, for example, by immersing the active carbon into the silylating agent or the solution. The contact is preferably performed at a temperature of 80 to 150° C. for the silylating agent in a liquid or solution form. Furthermore, stirring or ultrasonication is preferably performed for enhancing contact efficiency.

For the contact of the silylating agent in a liquid or solution form with the active carbon, a method comprising adding a basic compound together therewith is also preferable, because this method promotes the reaction of the silylating agent and the active carbon (see e.g., a method described in Carbon 42 (2004), 2113-2130).

The basic compound is preferably, but not particularly limited to, inorganic alkali salts (e.g., sodium hydroxide, potassium hydroxide, calcium hydroxide, and sodium bicarbonate) or amines (e.g., methylamine, ethylamine, propylamine, dimethylamine, diethylamine, dipropylamine, ammonia, and ammonium hydroxide). Among them, lower amines having 10 or less carbon atoms are more preferable, such as methylamine, ethylamine, propylamine, dimethylamine, diethylamine, and dipropylamine.

The vapor of the silylating agent that has been contacted with the active carbon in a tightly closed container can be removed from the silylated active carbon, for example, by the following procedures. (1) The pressure in the tightly closed container is reduced, and then the active carbon is heated at a temperature of approximately 80 to 450° C. (2) An inert gas such as argon is introduced into the tightly closed container, and the pressure reduction procedure is performed again. (3) The container is further filled with an inert gas, and then the silylated active carbon is taken out of the container. (4) The silylated active carbon is washed, if necessary, with water, an organic solvent, or the like.

The silylating agent in a liquid or solution form that has been contacted with the active carbon can usually be removed from the silylated active carbon by filtration. The silylated active carbon as obtained by the filtration may be washed, if necessary, with water, an organic solvent, or the like.

The silylating agent is generally contacted, with the active carbon, in an amount of, for example, but not particularly limited to, 0.001 to 1000 parts by weight, preferably 0.1 to 100 parts by weight, more preferably 0.1 to 10 parts by weight, per weight of the active carbon.

The silylated active carbon according to the present invention usually has a silylated functional group.

The silylated functional group of the silylated active carbon can be confirmed by various methods. For example, an approach can be used, such as X-ray spectroscopy or measurement using the curve of equilibrium moisture regain, described in Japanese Patent Laid-Open No. 9-77508, or ²⁹Si MAS NMR or ¹H-²⁹Si MAS NMR described in Carbon 43 (2005), 2554-2563. Moreover, according to the present method, the removal of the silylating agent used can also be confirmed.

Chemical shift of the silylated active carbon in a ¹H-²⁹Si MAS NMR spectrum may be shifted to higher magnetic field than that of silylating agent in a ²⁹Si MAS NMR spectrum. In the present invention, the silylated active carbon exhibits one or more characteristic peaks in a chemical shift region of −100 ppm to 0 ppm in a ¹H-²⁹Si MAS NMR spectrum.

The amount of silylated functional groups in the silylated active carbon is generally not lower than 0.01% by weight, preferably not lower than 0.05% by weight, more preferably not lower than 0.1% by weight, and generally not larger than 10% by weight, preferably not larger than 5% by weight, more preferably not larger than 3% by weight.

Herein, the amount of silylated functional groups is determined by ICP emission spectroscopy.

Examples of the noble metal according to the present invention include palladium, platinum, ruthenium, rhodium, iridium, osmium, and gold, and alloys and mixtures thereof.

Preferable examples of the noble metal include palladium, platinum, and gold. The noble metal is more preferably palladium.

For example, colloidal palladium may be used as the palladium (see e.g., Example 1 in Japanese Patent Laid-Open No. 2002-294301).

When a mixture of palladium and metal other than palladium is used as the noble metal, preferable examples of the metal other than palladium include gold and platinum.

In the present invention, the noble metal is usually used in an amount of 0.01 to 20% by weight, preferably 0.1 to 5% by weight, with respect to the total amount of the noble metal and the active carbon.

Supporting the noble metal by the silylated active carbon can be conducted, for example, by the following steps of allowing a noble metal compound to be supported by the silylated active carbon and then converting, to a noble metal, the noble metal compound supported by the active carbon.

The noble metal compound has the noble metal and can be any compound that is converted to a noble metal by reduction or the like, without limitations. The noble metal compound may be a complex.

The noble metal compound is preferably a palladium compound. Examples of the palladium compound include: tetravalent palladium compounds such as sodium hexachloropalladate (IV) tetrahydrate and potassium hexachloropalladate (IV); and divalent palladium compounds such as palladium (II) chloride, palladium (II) bromide, palladium (II) acetate, palladium (II) acetylacetonate, dichlorobis(benzonitrile)palladium (II), dichlorobis(acetonitrile)palladium (II), dichloro(bis(diphenylphosphino)ethane)palladium (II), dichlorobis(triphenylphosphine)palladium (II), dichlorotetraamminepalladium (II) dibromotetraamminepalladium (II), dichloro(cycloocta-1,5-diene)palladium (II), and palladium (II) trifluoroacetate. When the noble metal compound is a complex, an ammine complex such as Pd-tetraammine chloride is preferable.

The noble metal compound can be supported by a method conventionally known in the art such as impregnation.

The reduction can be performed by contacting a reducing agent such as hydrogen or ammonia gas with the noble metal compound. In this context, when an ammine complex is used as the noble metal compound, this noble metal compound supported by the active carbon, when subjected to heat treatment, generates ammonia gas through the thermal decomposition thereof. The noble metal compound is reduced with this ammonia gas as a reducing agent to obtain a noble metal.

The reduction can be performed at a temperature and a pressure appropriately set according to the kind of the noble metal compound or the reducing agent. For example, the reduction temperature is usually 100° C. to 500° C., preferably 200° C. to 350° C., for Pd-tetraammine chloride used as the noble metal compound.

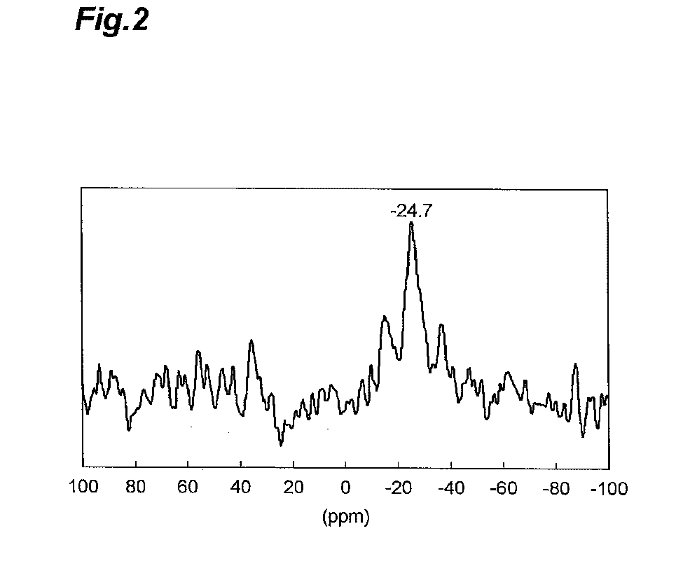

The titanosilicate according to the present invention is a generic name for silicate having tetracoordinated Ti (titanium atom) and has a porous structure. In the present invention, the titanosilicate means titanosilicate substantially having tetracoordinated Ti and shows that an UV-visible absorption spectrum of a wavelength region of 200 nm to 400 nm has the greatest absorption peak in a wavelength region of 210 nm to 230 nm (see e.g., FIGS. 2(d) and 2(e) in Chemical Communications, 1026-1027, (2002)). The UV-visible absorption spectrum can be measured by a diffuse reflection method using an UV-visible spectrophotometer equipped with a diffuse reflection attachment.

Examples of the titanosilicate according to the present invention include those having the following structures represented by the structural code specified by the International Zeolite Association (IZA).

TS-1 having an MFI structure

TS-2 having an MEL structure

Ti-ZSM-12 having an MTW structure

Ti-Beta having a BEA structure

Ti-MWW having an MWW structure

Ti-UTD-1 having a DON structure

Ti-MCM-68 having a DON structure

Examples of the TS-1, the TS-2, and the Ti-ZSM-12 include titanosilicate described in Zeolites 15, 236-242, (1995). Examples of the Ti-Beta include zeolite described in Journal of Catalysis 199, 41-47, (2001). Examples of the Ti-MWW include zeolite described in Chemistry Letters, 774-775, (2000). Examples of the Ti-UTD-1 include zeolite described in Zeolites 15, 519-525, (1995). Examples of the Ti-MCM-68 include zeolite described in Japanese Patent Laid-Open No. 2008-50186.

The titanosilicate according to the present invention may be titanosilicate such as a Ti-MWW precursor and Ti-YNU-1, which has a laminar structure and has a wider distance between the layers than that of an MWW structure.

The Ti-MWW precursor is titanosilicate having the laminar structure and forming Ti-MWW through dehydration condensation. The dehydration condensation can be performed, for example, by heating at a temperature of 250 to 800° C. Examples of the Ti-MWW precursor include zeolite described in Japanese Patent Laid-Open No. 2005-262164 or EP1731515A1.

Examples of the Ti-YNU-1 include titanosilicate described in Angewandte Chemie International Edition 43, 236-240, (2004).

The titanosilicate according to the present invention may be, for example, silylated titanosilicate. The silylated titanosilicate is preferable, because it has higher catalyst activities or selectivity. Examples of such titanosilicate include titanosilicate obtained by silylating the titanosilicate as described above with a silylating agent. Examples of the silylating agent for silylating the titanosilicate include 1,1,1,3,3,3-hexamethyldisilazane.

In the present invention, the titanosilicate is preferably titanosilicate having a pore composed of 12- or more membered oxygen ring (hereinafter, the present titanosilicate is referred to as “titanosilicate I”).

The pore is composed of Si—O or Ti—O bonds. The pores may be hemispherical pores called side pockets. Specifically, the pores do not have to penetrate a primary particle of the titanosilicate.

The “12- or more membered oxygen ring” means that the ring structure has 12 or more oxygen atoms in (a) the section of the narrowest place in the pores or (b) the entrance to the pores.

The titanosilicate I usually has pores of 0.6 nm to 1.0 nm in pore size.

In the present invention, the pore size means the diameter of (a) the section of the narrowest place in the pores or (b) the entrance to the pores.

The pore size herein is generally confirmed by analyzing an X-ray diffraction pattern of the titanosilicates.

The pore composed of an 12- or more membered oxygen ring in the titanosilicate are generally confirmed by analyzing an X-ray diffraction pattern or can be confirmed conveniently for a known structure by comparing the X-ray diffraction pattern with a known one.

Examples of the titanosilicate I include Ti-ZSM-12, Ti-Beta, Ti-MWW, Ti-MCM-68, Ti-UTD-1, and a Ti-MWW precursor.

The titanosilicate according to the present invention is more preferably titanosilicate that exhibits the following X-ray diffraction pattern:

Interplanar Spacing d
1.24±0.08 nm (12.4±0.8 Å)
1.08±0.03 nm (10.8±0.3 Å)
0.9±0.03 nm (9±0.3 Å)
0.6±0.03 nm (6±0.3 Å)
0.39±0.01 nm (3.9±0.1 Å)
0.34±0.01 nm (3.4±0.1 Å).

The X-ray diffraction is measured using a general X-ray diffractometer using copper Kα X-rays.

Examples of the titanosilicate that exhibits the X-ray diffraction pattern include a Ti-MWW precursor, Ti-YNU-1, Ti-MWW, and Ti-MCM-68.

The titanosilicate according to the present invention is further preferably a Ti-MWW precursor.

The Ti-MWW precursor can be prepared, for example, by the following methods 1 and 2.

Method 1: the method comprises heating, under pressure, a mixture containing a boron compound, a titanium compound, a silicon compound, a structure-directing agent, and water to obtain a laminar compound (also called an as-synthesized sample), and then being contacted with an aqueous strong acid solution under reflux conditions for removal of the structure-directing agent therefrom to obtain a Ti-MWW precursor (method described in, e.g., Japanese Patent Laid-Open No. 2005-262164).

Method 2: the method comprises mixing Ti-MWW, piperidine, and water, then heating the mixture under pressure, and washing the obtained compound with water (method described in Catalysis Today 117 (2006), 199-205).

In the method 1, the structure-directing agent is a nitrogen-containing compound that contributes to the formation of zeolite. Examples of the structure-directing agent include organic amines such as piperidine and hexamethyleneimine Examples of the aqueous strong acid solution include nitric acid. The contact of the laminar compound with the aqueous strong acid solution is preferably performed until the molar ratio of silicon to nitrogen (Si/N ratio) in the obtained compound is 21 or more.

The Ti-MWW precursor obtained by the method 2 has an Si/N ratio of 8.5 to 8.6 calculated from Si/Ti and Si/B ratios as a result of CHN elementary analyses described therein and thus has a higher nitrogen content than that of a usual Ti-MWW precursor. Such a Ti-MWW precursor can also be used as preferable titanosilicate in the present invention.

In the present invention, the titanosilicate can also be contacted in advance with hydrogen peroxide and then subjected to the reaction.

A hydrogen peroxide solution can be used as the hydrogen peroxide in the contact. The hydrogen peroxide solution usually has a hydrogen peroxide concentration ranging from 0.0001% by weight to 50% by weight. The hydrogen peroxide solution may be an aqueous solution or a solution obtained using a solvent other than water. The solvent other than water can be selected as a suitable one from among solvents contained in a solvent described below. The contact is usually performed at a temperature ranging from 0° C. to 100° C., preferably 0° C. to 60° C. The contact time differs depending on the concentration of hydrogen peroxide and is usually 10 minutes to 5 hours, preferably 1 hour to 3 hours.

The production method of the present invention is usually performed in a solvent comprising water, an organic solvent, or a mixture thereof. Examples of the organic solvent include alcohol, ketone, nitrile, ether, aliphatic hydrocarbon, aromatic hydrocarbon, halogenated hydrocarbon, ester, and mixtures thereof.

Examples of the aliphatic hydrocarbon include aliphatic hydrocarbons having 5 to 10 carbon atoms, such as hexane and heptane. Examples of the aromatic hydrocarbon include aromatic hydrocarbons having 6 to 15 carbon atoms, such as benzene, toluene, and xylene.

Examples of the alcohol include monohydric alcohol having 1 to 6 carbon atoms and glycol having 2 to 8 carbon atoms. The alcohol is preferably aliphatic alcohol having 1 to 8 carbon atoms, more preferably monohydric alcohol having 1 to 4 carbon atoms, such as methanol, ethanol, isopropanol, and t-butanol, even more preferably t-butanol.

Examples of the nitrile include: acetonitrile; nitrile having saturated aliphatic hydrocarbon, such as propionitrile, isobutyronitrile, and butyronitrile; and aromatic nitrile such as benzonitrile.

The organic solvent is preferably alcohol or nitrile from the viewpoint of catalyst activities and selectivity.

The organic solvent is more preferably linear or branched saturated aliphatic nitrile and aromatic nitrile, because it suppresses the formation of by-products. Examples of such a nitrile compound include: C2 to C4 alkylnitrile such as acetonitrile, propionitrile, isobutyronitrile, and butyronitrile; and benzonitrile. Acetonitrile is preferable.

When the mixture of water and an organic solvent is used as the solvent, the organic solvent-to-water weight ratio (organic solvent:water) is usually 90:10 to 0.01:99.99, preferably 50:50 to 0.01:99.99.

At too large a water ratio, propylene oxide may tend to be degraded due to ring-opening caused by its reaction with water, possibly resulting in decreased selectivity of propylene oxide. On the contrary, at too large an organic solvent ratio, solvent recovery cost may be increased.

In the production method of the present invention, the presence of a buffer salt in the solvent can prevent decrease in catalyst activities, further enhance catalyst activities, or improve the use efficiency of a source gas.

The buffer salt is usually added in an amount of 0.001 mmol/kg to 100 mmol/kg per kg of the solvent.

The buffer salt is preferably a salt having ammonium, alkylammonium, or alkylarylammonium.

Examples of the salt having ammonium, alkylammonium, or alkylarylammonium include buffer salts comprising 1) an anion selected from the group consisting of sulfate ion, hydrogen sulfate ion, carbonate ion, hydrogen carbonate ion, phosphate ions, hydrogen phosphate ion, dihydrogen phosphate ion, hydrogen pyrophosphate ion, pyrophosphate ions, halogen ion, nitrate ions, hydroxide ion, and C₁ to C₁₀ carboxylate ions and 2) a cation selected from the group consisting of ammonium, C₁ to C₂₀ alkylammonium, C₇ to C₂₀ alkylarylammonium, alkali metals, and alkaline-earth metals.

Examples of the C₁ to C₁₀ carboxylate ions include formate ion, acetate ion, propionate ion, butyrate ion, valerate ion, caproate ions, caprylate ion, capric acid ion, and benzoic acid ion.

Examples of the alkylammonium include tetramethylammonium, tetraethylammonium, tetra-n-propylammonium, tetra-n-butylammonium, and cetyltrimethylammonium. Examples of the alkali metal and alkaline-earth metal cations include lithium, sodium, potassium, rubidium, cesium, magnesium, calcium, strontium, and barium cations.

Preferable examples of the buffer salt include: ammonium salts of inorganic acids, such as ammonium sulfate, ammonium hydrogen sulfate, ammonium carbonate, ammonium hydrogen carbonate, diammonium hydrogen phosphate, ammonium dihydrogen phosphate, ammonium phosphate, ammonium hydrogen pyrophosphate, ammonium pyrophosphate, ammonium chloride, and ammonium nitrate; and ammonium salts of C₁ to C₁₀ carboxylic acids, such as ammonium acetate. Preferable examples of the ammonium salts include ammonium dihydrogen phosphate.

Instead of the buffer salt, a salt that generates an anion in a solvent may be contained in advance in the catalyst. For example, this method comprises allowing, for example, an ammine complex such as Pd-tetraammine chloride to be supported by the active carbon and then reducing the ammine complex while allowing ammonium ions to remain such that the anion of the buffer salt is formed during the production of propylene oxide.

In the production method of the present invention, a quinoid compound is preferably added, together with the titanosilicate and the catalyst, to the solvent, because the addition can further enhance propylene oxide selectivity.

Examples of the quinoid compound include a ρ-quinoid compound and a phenanthraquinone compound of the following formula (1):

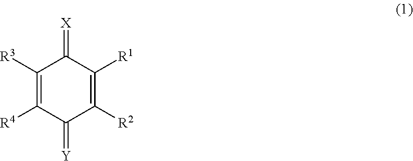

wherein R¹ and R² each independently represent a hydrogen atom, or R¹ and R² are combined, together with their carbon atoms to which R¹ and R² are bonded, to represent a benzene ring that may be substituted with an alkyl or hydroxyl group or a naphthalene ring that may be substituted with an alkyl or hydroxyl group; R³ and R⁴ each independently represent a hydrogen atom, or R³ and R⁴ are combined, together with their carbon atoms to which R³ and R⁴ are bonded, to represent a benzene ring that may be substituted with an alkyl or hydroxyl group or a naphthalene ring that may be substituted with an alkyl or hydroxyl group; and X and Y are the same or different from each other and represent an oxygen atom or an NH group.

Examples of the compound of the formula (1) include:

1) a quinone compound (1A) represented by the formula (1) wherein R¹, R², R³, and R⁴ are a hydrogen atom, and both X and Y are an oxygen atom;
2) a quinoneimine compound (1B) represented by the formula (1) wherein R¹, R², R³, and R⁴ are a hydrogen atom, and X and Y are an oxygen atom and an NH group, respectively; and
3) a quinonediimine compound (1C) represented by the formula (1) wherein R¹, R², R³, and R⁴ are a hydrogen atom, and X and Y are an NH group.

The quinoid compound of the formula (1) encompasses the following anthraquinone compound (2):

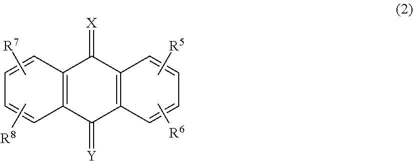

wherein X and Y are as defined in the formula (1); and R⁵, R⁶, R⁷, and R⁸ are the same or different from each other and represent a hydrogen atom, a hydroxyl group, or an alkyl group (e.g., C₁ to C₆ alkyl groups such as methyl, ethyl, propyl, butyl, and pentyl).

In the formulas (1) and (2), X and Y preferably represent an oxygen atom.

Examples of the quinoid compound include benzoquinone, naphthoquinone, anthraquinone, alkylanthraquinone compounds, polyhydroxyanthraquinone, ρ-quinoid compounds, and o-quinoid compounds.

Examples of the alkylanthraquinone compounds include: 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone, and 2-s-amylanthraquinone; and polyalkylanthraquinone compounds such as 1,3-diethylanthraquinone, 2,3-dimethylanthraquinone, 1,4-dimethylanthraquinone, and 2,7-dimethylanthraquinone. Examples of the polyhydroxyanthraquinone include 2,6-dihydroxyanthraquinone. Examples of the ρ-quinoid compounds include naphthoquinone and 1,4-phenanthraquinone. Examples of the o-quinoid compounds include 1,2-, 3,4-, and 9,10-phenanthraquinones.

Preferable examples of the quinoid compound include: anthraquinone; and 2-alkylanthraquinone compounds represented by the formula (2) wherein X and Y are an oxygen atom, R⁵ is an alkyl group substituted at position 2, and R⁶, R⁷, and R⁸ represent a hydrogen atom.

The quinoid compound can usually be used in an amount ranging from 0.001 mmol/kg to 500 mmol/kg per kg of the solvent.

The amount of the quinoid compound is preferably 0.01 mmol/kg to 50 mmol/kg.

The quinoid compound can also be prepared by oxidizing a dihydro form of the quinoid compound using oxygen or the like in the reaction system. For example, a hydrogenated quinoid compound such as hydroquinone or 9,10-anthracenediol is added to the solvent and oxidized with oxygen in the reactor to form the quinoid compound, which may then be used.

Examples of the dihydro form of the quinoid compound include compounds represented by the following formulas (3) and (4), which are dihydro forms of the compounds of the formulas (1) and (2):

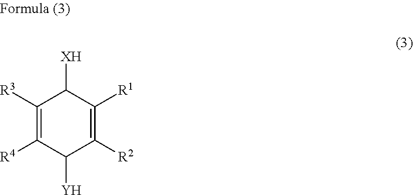

wherein R¹, R², R³, R⁴, X, and Y are as defined in the formula (1), and

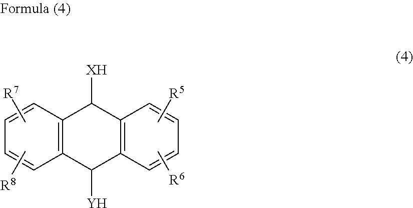

wherein X, Y, R⁵, R⁶, R⁷, and R⁸ are as defined in the formula (2).

In the formulas (3) and (4), X and Y preferably represent an oxygen atom.

Preferable examples of the dihydro form of the quinoid compound include dihydro forms corresponding to the preferable quinoid compounds.

Examples of the reaction in the production method of the present invention include fixed-bed reaction, stirred tank reaction, fluidized-bed reaction, moving-bed reaction, bubble column reaction, tubular reaction, and circular reaction.

In the production method, the partial pressure ratio between oxygen and hydrogen is usually oxygen:hydrogen=1:50 to 50:1, preferably 1:2 to 10:1.

At too high a partial pressure ratio between oxygen and hydrogen (oxygen/hydrogen), the rate of epoxy compound production may be decreased. At too low a partial pressure ratio between oxygen and hydrogen (oxygen/hydrogen), epoxy compound selectivity may be decreased due to increased by-products of paraffin.

In the present invention, the oxygen and hydrogen gases may be diluted with a gas for dilution. Examples of the gas for dilution include nitrogen, argon, carbon dioxide, methane, ethane, and propane. The gas for the dilution can be used at any concentration appropriately selected according to a reaction scale, the amount of the catalyst, etc, without limitations.

Examples of the oxygen as a raw material include oxygen gas and air. The oxygen gas used can be oxygen gas produced by an inexpensive pressure swing method or, if necessary, highly pure oxygen gas produced by cryogenic separation or the like.

In the production method, the propylene can be used in an amount appropriately selected according to the type thereof, reaction conditions, etc., and is usually used in an amount of 0.01 parts by weight, preferably 0.1 parts by weight, more preferably 1 part by weight, as the lower limit with respect to 100 parts by weight in total of the solvent and in an amount of 1000 parts by weight, preferably 100 parts by weight, more preferably 50 parts by weight, as the upper limit with respect to 100 parts by weight in total of the solvent.

In the production method, the titanosilicate can be used in an amount appropriately selected according to the type of the reaction and is usually used in an amount of 0.01 parts by weight, preferably 0.1 parts by weight, more preferably 0.5 parts by weight, as the lower limit with respect to 100 parts by weight in total of the solvent and in an amount of 20 parts by weight, preferably 10 parts by weight, more preferably 8 parts by weight, as the upper limit with respect to 100 parts by weight in total of the solvent.

In the production method, the noble metal and the active carbon can be used in amounts each appropriately selected according to the types thereof, reaction conditions, etc., and are preferably used in amounts that give the weight ratio between the noble metal and the titanosilicate in a range described later.

The amount of the noble metal is preferably 0.01 to 100 parts by weight, more preferably 0.1 to 20 parts by weight, relative to 100 parts by weight of the titanosilicate.

In the present invention, the reaction is usually performed at a temperature of 0° C., preferably 40° C., as the lower limit and at a temperature of 150° C., preferably 90° C., as the upper limit. At too low a reaction temperature, the reaction rate may be slowed down. By contrast, at too high a reaction temperature, by-products may be increased.

The reaction is usually performed at a pressure of 0.1 MPa to 20 MPa, preferably 1 MPa to 10 MPa, in terms of gage pressure.

The propylene oxide obtained by the present invention can be collected by a method known in the art such as separation by distillation. Unreacted olefin or solvents can be separated, for example, by distillation or membrane filtration.

### EXAMPLES

Hereinafter, the present invention will be described with reference to Examples. However, the present invention is not intended to be limited to these Examples.

### Elementary Analysis Method

1. The weights of Ti (titanium), Pd (palladium) and Si (silicon) were determined by ICP emission spectroscopy. Specifically, approximately 20 mg of a sample was weighed into a platinum crucible and covered with sodium carbonate, followed by fusion procedures using a gas burner. After the fusion, the content in the platinum crucible was dissolved by heating in pure water and nitric acid. Then, the solution was diluted with pure water, and then each element in this measurement solution was quantified using an ICP emission spectroscope (ICPS-8000 manufactured by Shimadzu Corp.).
2. The content of N (nitrogen) was measured by oxygen circulating combustion and TCD detection systems using SUMIGRAPH (manufactured by Sumika Chemical Analysis Service, Ltd.).

### X-Ray Powder Diffraction (XRD)

The X-ray powder diffraction pattern of a sample was determined using the following apparatus and conditions:

Apparatus: RINT2500V manufactured by Rigaku Corp.

### Source: Cu Kα X-rays

Output: 40 kV-300 mA
Scan range: 2θ=0.75 to 20°
Scan speed: 1°/min.

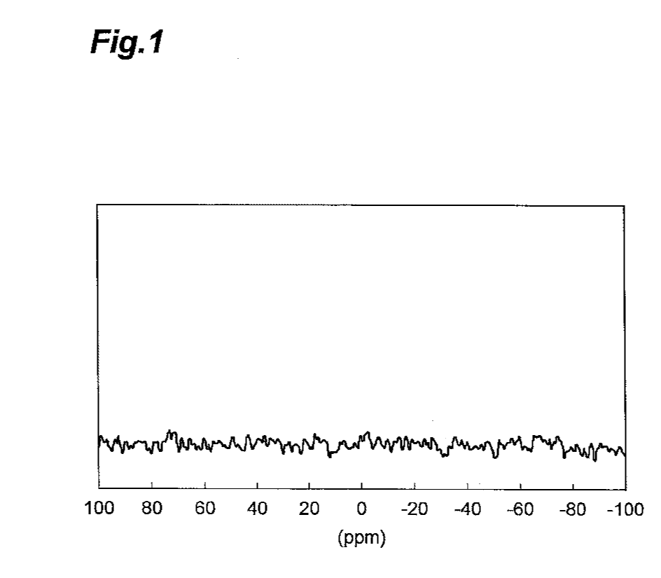

When the X-ray diffraction pattern was similar to that in FIG. 1 in EP1731515A1, the sample was determined to be a Ti-MWW precursor.

When the X-ray diffraction pattern was similar to that in FIG. 2 in EP1731515A1, the sample was determined to be Ti-MWW.

### UV-Visible Absorption Spectrum (UV-Vis)

A sample was well pulverized using an agate mortar and then pelletized (7 mmφ). The UV-visible absorption spectrum of this pellet was measured using the following apparatus and conditions:

Apparatus: diffuse reflection accessory (Praying Mantis manufactured by HARRICK Scientific Products)
Attachment: UV-visible spectrophotometer (manufactured by JASCO Corp. (V-7100))
Pressure: atmospheric pressure
Measurement value: reflectance
Data capture time: 0.1 sec.
Band width: 2 nm
Measurement wavelength: 200 to 900 nm
Slit height: half-open
Data capture interval: 1 nm
Baseline correction (reference): BaSO₄ pellet (7 mmφ)

When the UV-visible absorption spectrum of a wavelength region of 200 nm to 400 nm had the greatest absorption peak in a wavelength region of 210 nm to 230 nm, the sample was determined to be titanosilicate.

Gas chromatography analysis: HP5890 series II (manufactured by Agilent Technologies) was used.

### NMR Spectra

1H-29Si CPMAS NMR spectra of activated carbon and silylated activated carbon were measured at 119.23 MHz with Bruker AV-600 spectrometer and a CPMAS probe. Chemical shifts were referenced to an external standard of sodium 3-(trimethylsilyl)propionate-2,2,3,3-d₄ (1.445 pm). A spinning rate 3.0 kHz, recycle delay time of 5 sec. and 4000-20000 scans were taken. Magnetization transfer from protons to 29Si nuclei (cross-polarization) was achieved using a single contact time of 4 ms and a repetition time of 5 s.

29Si NMR spectra of silylating agent were measured in the absence of solvent at 119.23 MHz with Bruker AV-600 spectrometer. A spinning rate 3.0 kHz, recycle delay time of 5 sec. and 4000-20000 scans were taken. Chemical shifts were referenced to an external standard of sodium 3-(trimethylsilyl)propionate-2,2,3,3-d₄ (1.445 pm).

### Reference Example 1

Titanosilicate used in each Example and Comparative Example was prepared by the following method.

In an autoclave, 899 g of piperidine, 2402 g of pure water, 22.4 g of tetra-n-butyl orthotitanate (TBOT), 565 g of boric acid, and 410 g of fumed silica (cab-o-sil M7D) were dissolved with stirring at room temperature in an air atmosphere to prepare a gel (I). The obtained gel (I) was aged at 25° C. for 1.5 hours. Then, the autoclave was tightly closed, and the aged gel (I) was heated to 160° C. over 8 hours with stirring and then kept at this temperature for 120 hours for hydrothermal synthesis. After filtration of the obtained suspended solution, the obtained solid was washed with water until the pH of the filtrate was 10.4. Next, the solid was dried at 50° C. until no decrease in weight was seen, to obtain 564 g of solid (A).

To 75 g of the solid (A), 3750 mL of 2 M nitric acid and 9.5 g of TBOT were added, and the mixture was refluxed for 20 hours. After filtration of the obtained reaction mixture, the obtained solid was washed with water until the pH of the filtrate was around neutral. The solid was further vacuum-dried at 150° C. until no decrease in weight was seen, to obtain 62 g of a white powder (a).

This white powder (a) was confirmed from the UV-visible absorption spectrum to be titanosilicate and confirmed from the X-ray diffraction pattern to have a Ti-MWW precursor structure. The white powder (a) had a Ti content of 1.56% by mass and an Si/N ratio of 55.

Sixty (60) g of the white powder (a) was heated at 530° C. for 6 hours to obtain 54 g of solid (B) (Ti-MWW). The solid (B) was confirmed by X-ray diffraction pattern measurement to have an MWW structure. Furthermore, the same procedure as above was performed twice to obtain 162 g in total of Ti-MWW.

In an autoclave, 300 g of piperidine, 600 g of pure water, and 110 g of the solid (B) were dissolved with stirring at room temperature in an air atmosphere to prepare a gel (II). The gel (II) was aged at 25° C. for 1.5 hours. Then, the autoclave was tightly closed, and the aged gel (II) was heated to 160° C. over 4 hours with stirring and then kept at this temperature for 24 hours.

After filtration of the obtained suspended solution, the obtained solid was washed with water until the pH of the filtrate was around 9. Next, the solid was vacuum-dried at 150° C. until no decrease in weight was seen, to obtain 108 g of a white powder (b). As a result of measuring an X-ray diffraction pattern and an UV-visible absorption spectrum, this white powder (b) was confirmed to be a Ti-MWW precursor. The white powder (b) had a Ti content of 1.58% by mass and an Si/N ratio of 10.

Furthermore, the white powder (b) was treated at room temperature for 1 hour with 100 g (per 0.6 g of the white powder (b)) of a water/acetonitrile=20/80 (weight ratio) solution containing 0.1% by weight of hydrogen peroxide. After filtration, the solid was washed with 500 mL of water. The Ti-MWW precursor obtained by the hydrogen peroxide treatment was used as titanosilicate.

### Reference Example 2

A noble metal supported by silylated active carbon, used in Example 1 was prepared by the following method.

Ten (10) g of commercially available active carbon (powdery active carbon, manufactured by Wako Pure Chemical Industries Ltd.) was washed with 10 L of hot water (100° C.) and dried in a nitrogen stream at 150° C. for 6 hours (washed active carbon). The silicon content by ICP emission analysis was 0.21% by weight.

Six (6) g of the obtained washed active carbon was introduced into a glass container, then dried in a nitrogen stream at 160° C., and then allowed to cool. After cooling to room temperature, 50 mL of ethanol (dehydrated, manufactured by Wako Pure Chemical Industries Ltd.) and 9.2 g of dimethyldichlorosilane were added onto the active carbon, and the active carbon was left standing for 3 days. The active carbon thus left standing was separated by filtration and washed with 500 mL of ethanol, 2 L of water, and 5 L of hot water (100° C.) in this order. The active carbon thus washed was further dried at room temperature for 12 hours to obtain 7.7 g of silylated active carbon (hereinafter, this silylated active carbon is referred to as “silylated active carbon (I)”). The silicon content by ICP emission analysis was 0.50% by weight.

To a 1-L eggplant-shaped flask, 2.5 g of the silylated active carbon (I) and 200 mL of water were added, and the mixture was stirred in air at room temperature. To this suspension, 100 mL of an aqueous solution containing 0.20 mmol of colloidal Pd (manufactured by JGC Catalysts and Chemicals Ltd.) was gradually added dropwise in air at room temperature. After the completion of the dropwise addition, the suspension was further stirred in air at room temperature for 8 hours. After the completion of the stirring, the moisture was removed using a rotary evaporator, and the residue was vacuum-dried at 80° C. for 6 hours to obtain a noble metal supported on silylated active carbon (I) (hereinafter, this material composed of the silylated active carbon (I) and the noble metal is referred to as a “catalyst (I)”). The catalyst (I) had a Pd content of 1.24% by mass.

### Example 1

In a 0.5-L autoclave, 0.6 g of the titanosilicate and 0.016 g of the catalyst (I) were placed, and the following mixture gas at a rate of 22 NL/hr and the following anthraquinone solution at a rate of 108 mL/hr were supplied to the autoclave. Continuous reaction was performed under conditions involving a temperature of 60° C., a pressure of 0.8 MPa (gage pressure), and a residence time of 90 minutes by extracting the reaction mixture via a filter from the reactor.

Mixture gas composition: propylene/oxygen/hydrogen/nitrogen (volume ratio)=6.5/4.5/11/78

Anthraquinone solution: anthraquinone concentration: 0.7 mmol/kg and solvent: water/acetonitrile=20/80 (weight ratio)

Liquid and gas phases extracted after 5 hours into the reaction were analyzed by gas chromatography and consequently determined to have propylene oxide produced at a yield of 15.51 mmol/hr and propane produced at a yield of 0.36 mmol/hr.

### Reference Example 3

The procedure of contacting a silylating agent with active carbon described below was performed by a method described in Carbon 42 (2004), 2113-2130. Eight (8) g of the washed active carbon described in Reference Example 2 was introduced into a glass container, then dried in a nitrogen stream at 160° C., and then allowed to cool.

After cooling to room temperature, 40 mL of butylamine (manufactured by Kanto Chemical Co., Inc.) was added to the active carbon, followed by ultrasonication for 5 minutes. Then, the active carbon was subjected to heat treatment at 60° C. and allowed to cool after 1 hour. After cooling to room temperature, 180 mL of toluene (dehydrated, manufactured by Wako Pure Chemical Industries Ltd.) and 24 g of octyltrichlorosilane (manufactured by Shin-Etsu Chemical Co., Ltd.) were added to the active carbon, and the active carbon was left standing for 12 hours. After contacting it to octyltrichlorosilane, the silylated active carbon as obtained was separated by filtration, then washed with 500 mL of toluene, 500 mL of hexane, 500 mL of ethanol, 500 mL of acetone, 500 mL of ethanol/water=1/1 (volume ratio), 500 mL of acetone, and 5 L of hot water (100° C.) in this order, and further dried in a nitrogen stream at 150° C. for 6 hours to obtain 7.9 g of silylated active carbon (hereinafter, this silylated active carbon is referred to as “silylated active carbon (II)”). The silicon content by ICP emission analysis was 2.17% by weight.

To a 1-L eggplant-shaped flask, 3 g of the silylated active carbon (II) and 300 mL of water were added, and the mixture was stirred in air at room temperature. To this suspension, 100 mL of an aqueous solution containing 0.30 mmol of colloidal Pd (manufactured by JGC Catalysts and Chemicals Ltd.) was gradually added dropwise in air at room temperature. After the completion of the dropwise addition, the suspension was further stirred in air at room temperature for 8 hours. After the completion of the stirring, the moisture was removed using a rotary evaporator, and the residue was vacuum-dried at 80° C. for 6 hours, further washed with 5 L of hot water (100° C.), and dried in a nitrogen stream at 150° C. for 6 hours to obtain a noble metal supported on silylated active carbon (II) (hereinafter, this material composed of the silylated active carbon (II) and the noble metal is referred to as a “catalyst (II)”).

### Example 2

Propylene oxide production was performed in the same way as in Example 1 except that 0.02 g of the catalyst (II) was used instead of 0.016 g of the catalyst (I). Liquid and gas phases extracted after 5 hours into the reaction were analyzed by gas chromatography and consequently determined to have propylene oxide produced at a yield of 14.91 mmol/hr and propane produced at a yield of 0.67 mmol/hr.

### Comparative Example 1

The same procedure as in Example 2 was performed except for using the catalyst which was made from the washed active carbon described in Reference Example 2 instead of the silylated active carbon (II). Liquid and gas phases extracted after 5 hours into the reaction were analyzed by gas chromatography and consequently determined to have propylene oxide produced at a yield of 13.15 mmol/hr and propane produced at a yield of 0.31 mmol/hr.

### INDUSTRIAL APPLICABILITY

According to the present invention, propylene oxide can be produced efficiently.

1. A method for producing propylene oxide which comprises reacting propylene, oxygen and hydrogen, in the presence of a titanosilicate and a noble metal supported on silylated active carbon, in a solvent.

2. The production method according to claim 1, wherein the titanosilicate has a pore composed of 12- or more membered oxygen ring.

3. The production method according to claim 1, wherein the titanosilicate is a crystalline titanosilicate having an MWW structure, or a Ti-MWW precursor.

4. The production method according to claim 1, wherein the titanosilicate has an X-ray diffraction pattern reproduced in the form of interplanar spacings d of
1.24±0.08 nm,
1.08±0.03 nm,
0.9±0.03 nm,
0.6±0.03 nm,
0.39±0.01 nm, and
0.34±0.01 nm.

5. The production method according to claim 1, wherein the solvent comprises an organic solvent.

6. The production method according to claim 5, wherein the organic solvent comprises at least one selected from the group consisting of alcohol, ketone, nitrile, ether, aliphatic hydrocarbon, aromatic hydrocarbon, halogenated hydrocarbon, and ester.

7. The production method according to claim 5, wherein the organic solvent is acetonitrile.

8. The production method according to claim 5, wherein the solvent is a mixture of an organic solvent and water having an organic solvent-to-water weight ratio of 90:10 to 0.01:99.99.

9. The production method according to claim 1, wherein the solvent contains a salt having an ammonium, alkylammonium, or alkylarylammonium ion.

10. The production method according to claim 9, wherein the salt comprises 1) an anion selected from sulfate ion, hydrogen sulfate ion, carbonate ion, hydrogen carbonate ion, phosphate ion, hydrogen phosphate ion, dihydrogen phosphate ion, hydrogen pyrophosphate ion, pyrophosphate ion, halogen ion, nitrate ion, hydroxide ion, and carboxylate ions having 1 to 10 carbon atoms and 2) a cation selected from ammonium ion, alkylammonium ions, and alkylarylammonium ions.

11. The production method according to claim 1, wherein the solvent contains a quinoid compound or a dihydro form thereof.

12. The production method according to claim 11, wherein the quinoid compound is a phenanthraquinone compound or a compound represented by formula (1): 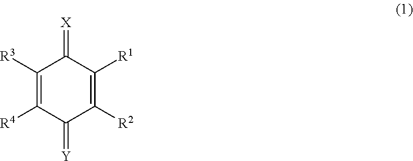
wherein R¹ and R² each independently represent a hydrogen atom, or R¹ and R² are combined, together with their carbon atoms to which R¹ and R² are bonded, to represent a benzene ring that may be substituted with an alkyl or hydroxyl group or a naphthalene ring that may be substituted with an alkyl or hydroxyl group; R³ and R⁴ are combined, together with their carbon atoms to which R³ and R⁴ are bonded, to represent a benzene ring that may be substituted with an alkyl or hydroxyl group or a naphthalene ring that may be substituted with an alkyl or hydroxyl group; and X and Y each independently represent an oxygen atom or an NH group.

13. The production method according to claim 12, wherein the quinoid compound is a phenanthraquinone compound or a compound represented by formula (2): 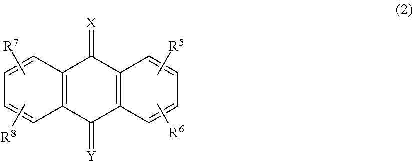
wherein X and Y each independently represent an oxygen atom or an NH group; and R⁵, R⁶, R⁷, and R⁸ each independently represent a hydrogen atom, a hydroxyl group, or an alkyl group.

14. The production method according to claim 12, wherein X and Y are oxygen atoms.
